(19) United States
(12) Patent Application Publication
(10) Pub. No.: US 2026/0071158 A1
(43) Pub. Date: Mar. 12, 2026

(54) CONTINUOUS ALGAE CULTIVATION PHOTOREACTION DEVICE

(71) Applicant: SWISCARE TECHNOLOGY CO., LTD., New Taipei City (TW)

(72) Inventor: Erik CHEN, New Taipei City (TW)

(21) Appl. No.: 18/829,857

(22) Filed: Sep. 10, 2024

(51) Int. Cl. C12M 1/00; C12M 1/34

(52) U.S. Cl. CPC C12M 21/02; C12M 23/38; C12M 29/18; C12M 31/08; C12M 41/34

(57) ABSTRACT

The present invention provides a continuous algae cultivation photoreaction device, which includes: a base, a light-transmitting tank, an outer cover casing, and an upper cover. The device mainly involves placing algae and liquid inside the light-transmitting tank, and using light emitted by an omnidirectional lighting lamp embedded in the base to irradiate the algae. Moreover, the light also irradiates light reflecting layers fitted to the inner sides of the outer cover casing, whereby reflecting and scattering effects of the light occur, which can then further irradiate a wider area of the algae and achieving the object of substantially improving photoreaction efficiency thereof.

### BACKGROUND OF THE INVENTION

### Field of the Invention

The present invention relates to a continuous algae cultivation photoreaction device, and more particularly to a device structure that substantially improves photoreaction efficiency of algae.

### Description of the Prior Art

Environmentally friendly carbon-reducing energy has always been an issue of global concern. Hence, in recent years, many methods have arisen to generate green energy, such as through wind power, water power, solar energy, photoreaction, etc., among which the photoreaction method mainly involves cultivating a large amount of algae and performing photosynthesis through sunlight, causing the production of oxygen and a variety of high value-added active substances.

The majority of current photoreaction methods involve placing algae in a light-transmitting tank situated outdoors, and allowing sunlight to irradiate the algae inside the light-transmitting tank, to steadily proceed with producing a photosynthesis effect. However, the conditions for solar irradiation include not only proceeding during the day, meaning a substantial reduction in reaction efficiency on cloudy days or when the sunlight level is low; furthermore, the angle of sunlight irradiation will vary during the day, but the angle position of the light-transmitting tank is fixed and cannot be adjusted. Consequently, a lot of the algae in the light-transmitting tank will not be irradiated by the sunlight. In addition, because the algae will continue to grow, the expanding area of algae will impact the photosynthesis effect. Moreover, current methods require removing all the alga and cleaning the light-transmitting tank before cultivating a new batch of algae, which is not only time-consuming, cleaning the light-transmitting tank is also quite difficult. Hence, the inventor of the present invention proposes to resolve and surmount existent technical difficulties to eliminate the aforementioned shortcomings of the prior art.

### SUMMARY OF THE INVENTION

The main object of the present invention lies in: assembling a continuous algae cultivation photoreaction device from a base, a light-transmitting tank, an outer cover casing, and an upper cover; the function of which mainly involves placing algae and liquid inside the light-transmitting tank, and then irradiating the algae with continuance light emitted by an omnidirectional lighting lamp embedded in the base. Moreover, the emitted light also irradiates light reflecting layers fitted to inner sides of the outer cover casing, whereupon light reflecting and scattering effects occur, which can then further irradiate a wider area of algae and thereby achieve the object of substantially improving photoreaction efficiency thereof.

In order to achieve the above-described object, a gas detector and a gas refiner are additionally connected to a gas intake tube fitted in the base.

In order to achieve the above-described object, a filtering device is additionally connected to a water outlet tube.

In order to achieve the above-described object, the lower side of the base is additionally provided with a plurality of corresponding protruding pieces.

In order to achieve the above-described object, a fixing hole provided in the outer cover corresponds to an opening provided in the light-transmitting tank.

In order to achieve the above-described object, an omnidirectional lighting lamp is additionally embedded into the fixing hole of the outer cover casing.

In order to attain the above-described object, a gas detector is additionally connected to a discharge tube configured in the upper cover.

In order to achieve the above-described object, a plurality of corresponding indentations are additionally provided in the upper side of the upper cover.

To enable a further understanding of said objectives, structures, characteristics, and effects, as well as the technology and methods used in the present invention and effects achieved, a brief description of the drawings is provided below followed by a detailed description of the preferred embodiments.

### BRIEF DESCRIPTION OF THE DRAWINGS

FIG. 1 is a three-dimensional external view of the present invention.

FIG. 2 is an exploded three-dimensional view of the present invention.

FIG. 3 is a first schematic view of a preferred embodiment of the present invention.

FIG. 4 is a second schematic view of the preferred embodiment of the present invention.

FIG. 5 is a third schematic view of the preferred embodiment of the present invention.

FIG. 6 is a fourth schematic view of the preferred embodiment of the present invention.

### DETAILED DESCRIPTION OF THE PREFERRED EMBODIMENTS

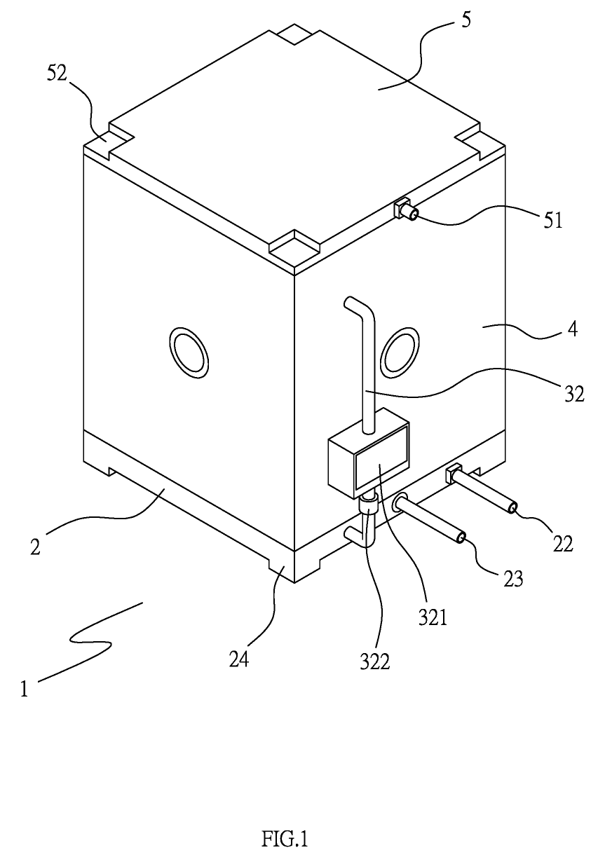

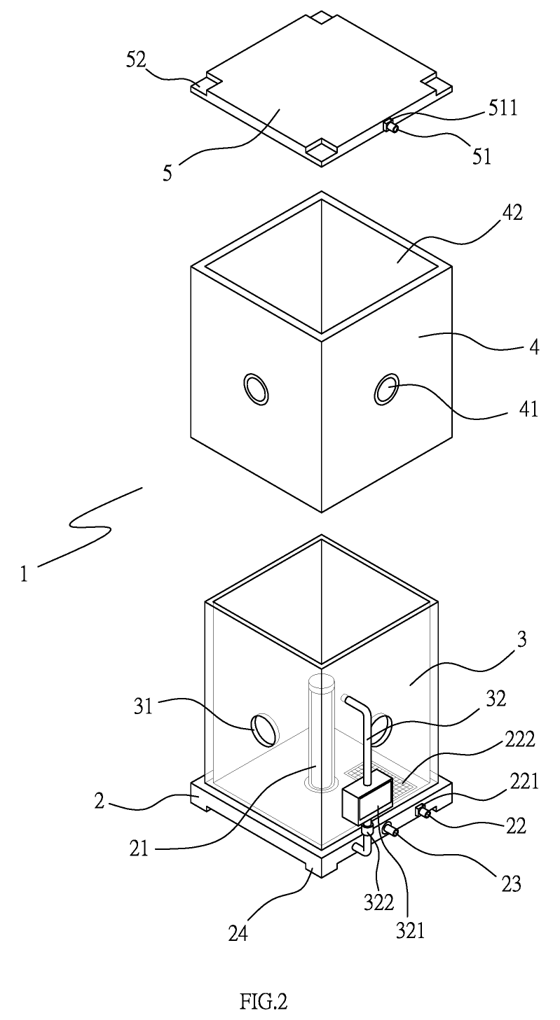

Referring to FIGS. 1, 2, 3, 4, 5 and 6, which show a three-dimensional external view, a three-dimensional exploded view, and schematic views of a preferred embodiment of the present invention, and it can be clearly seen from the drawings that a continuous algae cultivation photoreaction device 1 of the present invention mainly comprises:

a base 2, an upper side of which has at least one omnidirectional lighting lamp 21 embedded therein, with a gas intake tube 22 and a nutrient solution replenishing tube 23 connected and installed in the outer side edge of the base 2;

a light-transmitting tank 3, which is mounted on the base 2, and has at least one opening 31 provided therein, one end of a circulation tubing 32 is further connected and installed to the light-transmitting tank 3, and the other end of the circulation tubing 32 is connected to the base 2;

an outer cover casing 4, which is cover-mounted on the exterior of the light-transmitting tank 3, and has at least one fixing hole 41 provided therein, a light reflecting layer 42 is further fitted to the inner side surface of the outer cover casing 4; and

an upper cover 5, which is mounted on the light-transmitting tank 3, and one side of the upper cover 5 is fitted with at least one discharge tube 51.

The gas intake tube 22 fitted in the base 2 is additionally connected to a gas detector 221 and a gas refiner 222.

A collection filter 321 and a cut-off valve 322 are additionally connected to the circulation tubing 32.

The lower side of the base 2 is additionally provided with a plurality of corresponding protruding pieces 24.

The fixing holes 41 in the outer cover casing 4 correspond to the openings 31 in the light-transmitting tank 3.

The fixing holes 41 in the outer cover casing 4 can be used to embed the omnidirectional lighting lamp 21 therein.

A gas detector 511 is additionally connected to the discharge tube 51 of the upper cover 5.

A plurality of corresponding indentations 52 are additionally configured in the upper side of the upper cover 5.

Operating the continuous algae cultivation photoreaction device 1 of the present invention mainly involves first placing algae A and liquid B inside the light-transmitting tank 3 and covering with the upper cover 5, thereby sealing the light-transmitting tank 3 to form a gastight space. Finally the algae A is irradiated with continuance light emitted by the omnidirectional lighting lamp 21 embedded in the base 2 to achieve the object of causing a photoreaction in the algae A.

In accordance with the above, when the omnidirectional lighting lamp 21 is emitting light, in addition to directly irradiating the algae A in the vicinity of the omnidirectional lighting lamp 21, light reflecting and scattering effects occur after the light irradiates the light reflecting layers 42 fitted to the inner side surfaces of the outer cover casing 4, thereby enabling the light to produce a more widespread and multi-angle irradiation effect to achieve the object of irradiating a larger area of the algae A and substantially improving photoreaction efficiency thereof.

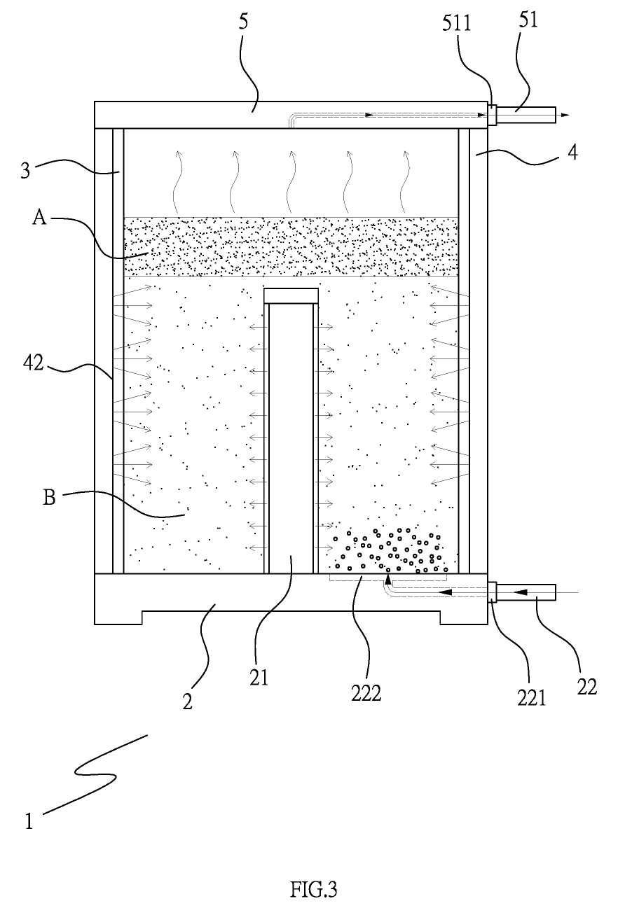

Further, while proceeding with the photoreaction operation, as depicted in FIG. 3, the gas intake tube 22 in the base 2 transmits carbon dioxide or other gases required for the photoreaction. Finally, the gas refiner 222 refines the gases before entering the light-transmitting tank 3, to enable quick absorption by the algae A. The gas detector 221 additionally connected to the gas intake tube 22 is used to sense and monitor the carbon dioxide being transmitted through the gas intake tube 22 or other related gas data, such as concentration and transmission quantity.

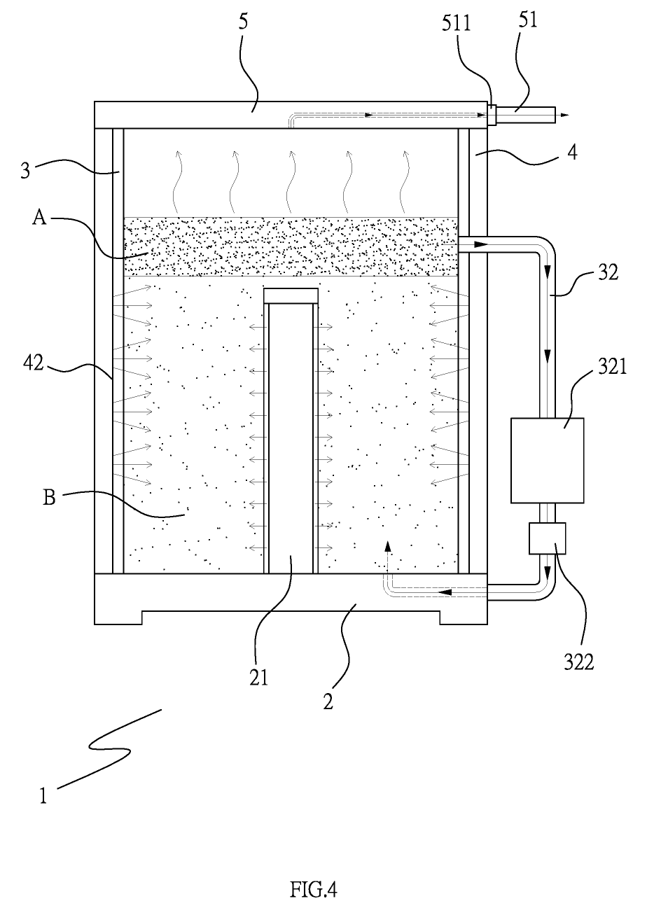

Furthermore, because the algae A will continue to grow, and the expanding area of the algae A will impact the photoreaction effect; thus, as shown in FIG. 4. the circulation tubing 32 connected and installed to the light-transmitting tank 3 is used to first discharge the algae A, which is then filtered through the collection filter 321 of the circulation tubing 32. Finally, the left over liquid B flows back through the other end of the circulation tubing 32 into the light-transmitting tank 3. Accordingly, in addition to enabling effective control of the amount and area of the algae A, the continuous algae cultivation photoreaction device 1 of the present also enables continuous recycling of the liquid B.

Further, in order to ensure normal growth of the algae A and prevent the algae A from dying, the nutrient solution replenishing tube 23 connected and installed to the outer side of the base 2 is used to replenish a relevant nutrient solution required by the algae A or to add substances required for the liquid B.

In addition, because oxygen or other gases are produced by the photoreaction when the omnidirectional lighting lamp 21 is irradiating the algae A, thus, the discharge tube 51 fitted to one side of the upper cover 5 is used to capture the oxygen or other gases produced. Moreover, the gas detector 511 additionally connected to the discharge tube 51 is used to sense and monitor related gas data, such as concentration and transmission quantity.

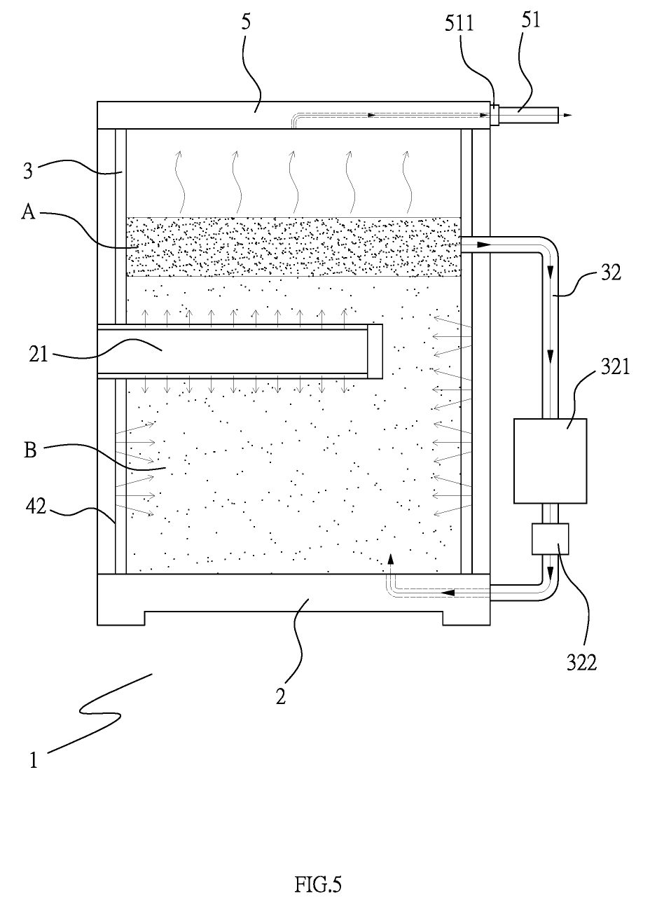

Referring to FIG. 4, which shows that the omnidirectional lighting lamp 21 is primarily embedded into the base 2 and positioned in the lower side of the light-transmitting tank 3 to carry out irradiation; however, as shown in FIG. 5, the omnidirectional lighting lamp 21 can also be embedded into the fixing hole 41 of the outer cover casing 4, thereby positioning the omnidirectional lighting lamp 21 in the side of the light-transmitting tank 3 to carry out irradiation. Basically, there are no specific restrictions to the installed position of the omnidirectional lighting lamp 21. The main consideration being that the omnidirectional lighting lamp 21 is positioned according to the height position of the algae A in the light-transmitting tank 3, i.e., the required position height of the omnidirectional lighting lamp 21 is set to achieve the preferred optimum irradiation effect of the algae A. It is understood that a plurality of the omnidirectional lighting lamps 21 can be concurrently embedded into the base 2 and the fixing holes 41 in the outer cover casing 4.

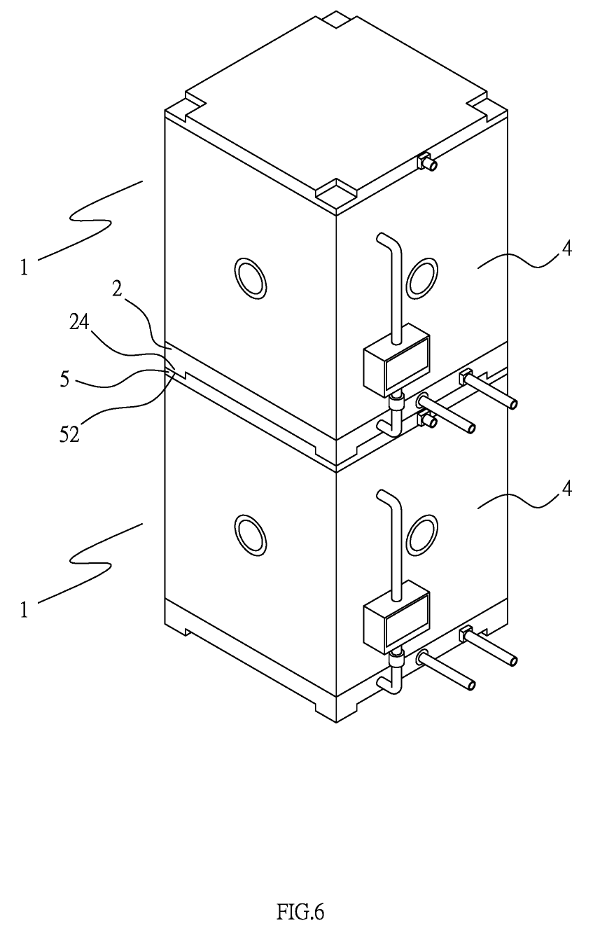

In addition, referring to FIG. 6, another continuous algae cultivation photoreaction device 1 can be embedded into the indentations 52 configured in the upper cover 5 by means of the protruding pieces 24 configured in the lower side of the base 2, thereby constructing a stacked configuration of a plurality of the continuous algae cultivation photoreaction devices 1. Accordingly, in addition to substantially reducing the plane area space required for placement of the plurality of the continuous algae cultivation photoreaction devices 1, such a stackable design can also further facilitate the centralized management of a plurality of the continuous algae cultivation photoreaction devices 1.

In conclusion, compared to the various existing shortcomings and unsolvable problems of photoreaction devices of the prior art, the continuous algae cultivation photoreaction device 1 of the present invention with the distinctive design of the base 2, the light-transmitting tank 3, the outer cover casing 4, and the upper cover 5 provides a sealed environment for the algae A placed therein to receive more widespread exposure to light irradiation. Moreover, the omnidirectional lighting lamp(s) 21 can be set at the optimum angle according to requirements, substantially improving photoreaction efficiency of the algae A. In addition, the circulation tubing 32 connected and installed to the light-transmitting tank 3 not only enables effective control of the amount and area of the algae A but also continuous recycling of the liquid B. Furthermore, the protruding pieces 24 configured in the lower side of the base 2 and the indentations 52 configured in the upper cover 5 enable constructing a stacked configuration of a plurality of the continuous algae cultivation photoreaction devices 1, thus further achieving the objects of maximizing space utilization and facilitating centralized management of the plurality of continuous algae cultivation photoreaction devices 1.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A continuous algae cultivation photoreaction device, comprising:
a base, an upper side of which has at least one omnidirectional lighting lamp embedded therein, with a gas intake tube and a nutrient solution replenishing tube connected and installed in an outer side edge of the base;
a light-transmitting tank, which is installed on the base and has at least one opening provided therein, one end of a circulation tubing is further connected and installed to the light-transmitting tank, and the other end of the circulation tubing is connected to the base;
an outer cover casing, which is cover-mounted on an exterior of the light-transmitting tank and has at least one fixing hole provided therein, a light reflecting layer is further fitted to the inner side surface of the outer cover casing; and
an upper cover, which is mounted on the light-transmitting tank, and one side of the upper cover is fitted with at least one discharge tube.

2. The continuous algae cultivation photoreaction device according to claim 1, wherein a gas detector and a gas refiner are additionally connected to a gas intake tube fitted in the base.

3. The continuous algae cultivation photoreaction device according to claim 1, wherein a collection filter and a cut-off valve are additionally connected to the circulation tubing.

4. The continuous algae cultivation photoreaction device according to claim 1, wherein the lower side of the base is additionally provided with a plurality of corresponding protruding pieces.

5. The continuous algae cultivation photoreaction device according to claim 1, wherein fixing holes provided in the outer cover correspond to openings provided in the light-transmitting tank.

6. The continuous algae cultivation photoreaction device according to claim 1, wherein the fixing holes in the outer cover casing are used to embed the omnidirectional lighting lamp therein.

7. The continuous algae cultivation photoreaction device according to claim 1, wherein a gas detector is additionally connected to a discharge tube configured in the upper cover.

8. The continuous algae cultivation photoreaction device according to claim 1, wherein a plurality of corresponding indentations are additionally provided in the upper side of the upper cover.
